(19) United States
(12) Patent Application Publication
(10) Pub. No.: US 2005/0079136 A1
(43) Pub. Date: Apr. 14, 2005

(54) Aerosol formulations of delta tetrahydrocannabinol

(76) Inventors: Austen John Woolfe, Essex (GB); Alan Keith Langford, Hertfordshire (GB)

Correspondence Address: IVAX CORPORATION, 4400 BISCAYNE BOULEVARD, MIAMI, FL, 33137, US

(21) Appl. No.: 10/483,655

(22) PCT Filed: Jul. 10, 2002
(86) PCT No.: PCT/GB02/03161

Related U.S. Application Data
(60) Provisional application No. 60/305,021, filed on Jul. 10, 2001.

(51) Int. Cl. A61K 9/00; A61L 9/04; A61K 31/353

(52) U.S. Cl. 424/45; 514/454

(57) ABSTRACT

The application discloses an aerosol formulation comprising Δ⁸ Tetrahydrocannabinol for use as a medicine, and the use of Δ⁸ Tetrahydrocannabinol to treat a condition selected from pain, appetite loss, multiple sclerosis and asthma.

### FIELD OF THE INVENTION

The invention is directed to the therapeutic use of Δ⁸ Tetrahydrocannabinol (Δ⁸ THC). In particular, the invention provides Δ⁸ THC formulations suitable for administration to the buccal or nasal mucosa or the pulmonary airways. Such Δ⁸ THC formulations are useful for the reduction, elimination or prevention of pain associated with any medical condition; the stimulation of appetite; the reduction, elimination or prevention of nausea; the reduction, elimination or prevention of vomiting (antiemetic properties); the relaxation of muscle tissue (e.g., for the treatment of multiple sclerosis).

### SUMMARY OF THE RELATED ART

Currently there is much interest in the possible medical use of Cannabis or its natural constituents. In Great Britain, for example, two House of Lords reports from 1999 and 2001 have both recommended further investigation as to whether the anecdotal (i.e., not scientifically proved) reports from certain patients with multiple sclerosis and other long term painful or debilitating diseases have a genuine basis.

Cannabis use is centuries old, particularly in China and India, although the abuse (mostly in the West) is of more recent origin and dates back only about 100 years.

There have been many arguments as to the dangers of Cannabis and its addictive potential, however a general consensus seems to be growing that it is probably no worse than tobacco in terms of addiction although there is a potential for longer term psychosis if large doses are taken for the immediate “high”. The common method of taking Cannabis is smoking, but this gives rise to similar bad effects on the lung from tars and other components as for tobacco.

Currently there are three approaches to the investigation of possible medical uses for cannabinoids (the name for the group of “active” molecules in Cannabis).

One is to try to standardize an extract from a plant or mixtures of plants. Much of the current work both in the UK and US is based on the use of a “Cannabis Oil” extracted from plants. This contains a mixture of natural molecules, some of which are at present not characterized. The extract must be standardized which is difficult to achieve even in rigorously controlled growing conditions and it is very difficult if not impossible to purify the active constituents away from plant materials such waxes, sterols etc.

The second is to try to develop new synthetic molecules based on the structures of the natural cannabinoids hopefully without some of the possible psychotropic side effects. The synthesis of new molecules is being investigated by a number of academic centers but is extremely costly to complete and bring to market. The generally accepted cost to carry out all the chemistry, pharmacology, clinical trials etc. to bring a new drug to market is usually quoted at about $300 million and this by no means guarantees success.

The third is to synthesize synthetic equivalents of some of the natural cannabinoid molecules. The main active constituent of Cannabis is now known to be THC (tetrahydrocannabinol) with two other major components Cannabidiol and Cannabinol depending on the plant used and the growing conditions.

There are then many other minor components some of which have been identified and some of which have not. These structures are shown below.

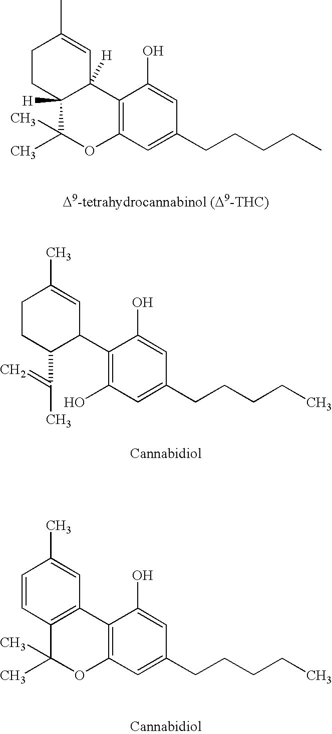

A major problem associated with the medicinal use of cannabinoids entails the method for administering said cannabinoids. Smoking Cannabis leaves or resin for medical use would not be acceptable in many countries e.g., UK, as it is not standardized, difficult to control the dosage and would result in similar tars etc., depositing in the lung as from tobacco smoking.

There are some current trials using capsules of Cannabis extracts or its synthetic components but these are known to be less than desirable as cannabinoids are rapidly metabolised in the body when given orally into the stomach (so called “First Pass Metabolism”) and large doses are needed to get possible active molecules into the blood stream in adequate amounts. This leaves large amounts of metabolites, some of which must have clinical activity of some sort and may well give rise to some of the unwanted side effects.

Others are using a standardized extract given under the tongue in the mouth where the active components are absorbed directly into the veins in the mouth so avoiding the “First Pass Metabolism”, they use a specially formulated spray to dose the drug.

Still others have tried similar approaches. While mixtures of active molecules were produced, it was impossible to remove all the associated plant material, which was of a waxy nature. This would make them unsuitable for administration directly into the lungs as the removal of waxy material from the lungs would be problematic and may well lead to a build up of wax in the lung with all the long term problems and dangers this may involve.

One possible approach to the problem entails the possibility of using chemically synthesized molecules or mixtures of the naturally occurring cannabinoids. This is because there is some limited toxicity data already available on such compounds. For example, Abrahamov, et al., (Life Sciences 56: 2097-2102, -1995 and U.S. Pat. No. 5,605,928) have shown promising results using a synthetic version of the THC in children with cancer where the incidence of nausea was greatly reduced with no significant side effects.

This molecule is called Δ⁸ THC in comparison the naturally occurring Δ⁹ THC, which as mentioned earlier, is the main naturally occurring active constituent of Cannabis. The structures are shown below and the two molecules can be seen to differ only by the position of a double bond from 8 to 9.

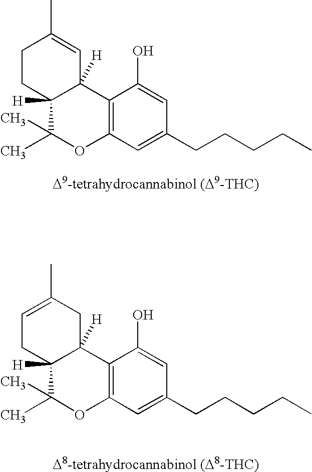

Δ⁸ THC is reportedly easier to synthesize the Δ⁹ THC. It exists as an oil at ambient temperature.

The literature has many anecdotal references to possible medicinal uses of Cannabis, for example: Relief of Pain (post operatively, Oncological, Phantom Limb etc), Multiple Sclerosis, Anti-nausea, Appetite. Stimulation, Asthma etc.

Pain relief in terminal oncology is now widely accepted to be the main concern of the physician and the main component of this is morphine normally given as delayed release tablets (or by injection or infusion). In the terminal stages of the disease, it often becomes difficult for the patient to swallow, either due to GI tract obstruction or an associated nausea caused by the disease or by some of the anti-cancer treatments, and so an aerosol treatment directly into the lungs might well be of significant value.

The present invention addresses such problems associated with medicinal cannabinoid administration by providing an aerosol formulation where the principle active medicament is Δ⁸ Tetrahydrocannabinol.

### SUMMARY OF THE INVENTION

The present invention provides aerosol formulations for the medicinal administration of Δ⁸ Tetrahydrocannabinol. In a second aspect, the invention provides a method for treating patients to alleviate the symptoms associated with a number of disease, states using Δ⁸ Tetrahydrocannabinol.

### DETAILED DESCRIPTION OF THE PREFERRED EMBODIMENTS

The present invention provides Δ⁸ Tetrahydrocannabinol formulations and methods for treating patients to alleviate the symptoms associated with a number of disease states. Therefore, an object of the invention is (a) the reduction, elimination or prevention of pain associated with any medical condition; (b) the stimulation of appetite; (c) the reduction, elimination or prevention of nausea; (d) the reduction, elimination or prevention of vomiting (antimetic properties); (e) the relaxation of muscle tissue (e.g. for the treatment of multiple sclerosis).

The active medicament for these formulations and methods is Δ⁸ Tetrahydrocannabinol (Δ⁸ THC).

The present invention provides aerosol formulations for the medicinal administration of Δ⁸ Tetrahydrocannabinol and novel medicinal uses of Δ⁸ Tetrahydrocannabinol. The term Δ⁸ Tetrahydrocannabinol (Δ⁸ THC) designates Δ⁸ Tetrahydrocannabinol and prodrugs (hereinafter collectively designated as “Δ⁸ THC moieties”).

According to one aspect, the present invention provides the use of Δ⁸ Tetrahydrocannabinol in the manufacture of an aerosol formulation for medicinal administration to a patient from an aerosol delivery device.

According to an alternative aspect, the present invention provides a method of treating a mammal suffering from a condition indicating treatment with a Δ⁸ Tetrahydrocannabinol, which comprises administering an aerosolized aerosol formulation containing a therapeutically effective amount of Δ⁸ Tetrahydrocannabinol to the mammal.

The condition may be any medical-condition indicating treatment with Δ⁸ Tetrahydrocannabinol, for example a condition selected from pain, nausea, vomiting, appetite loss, multiple sclerosis and asthma.

In one embodiment of the invention, the patient (or mammal) may be a cancer patient undergoing chemotherapy, and the condition is selected from pain, nausea, vomiting and appetite loss.

Particular mention may be made of the case where the condition is pain associated with phantom limb syndrome.

Particular mention may also be made of the case where the condition is appetite loss associated with anorexia nervosa.

In one embodiment, the aerosol formulation is for administration to the lungs of the patient.

In another embodiment, the aerosol formulation is for administration to the buccal or nasal mucosa of the patient.

According to another aspect, the present invention provides an aerosol delivery device containing an aerosol formulation comprising Δ⁸ Tetrahydrocannabinol.

According to yet another aspect, the present invention provides an aerosol formulation for use in an aersol delivery device, which comprises Δ⁸ Tetrahydrocannabinol.

Preferably, the aerosol formulation further comprises a propellant.

The propellant is preferably selected from 1,1,1,2-tetrafluoroethane (HFA 143a) and 1,1,1,2,3,3,3-heptafluoropropane (HFA 227).

Preferably, the aerosol formulation further comprises ethanol as a solvent.

For inhalation, the formulations of the present invention may be delivered via any inhalation methods known to those skilled in the art. Such inhalation methods and devices include, but are not limited to, metered dose inhalers with propellants such as CFC or HFA or propellants that are physiologically and environmentally acceptable. Other included devices are breath-operated inhalers, multidose dry powder inhalers and aerosol nebulizers. One preferred way of administering the formulations of the invention is by using conventional actuators. The term “actuator” as used in the present invention includes all types of actuators presently available including but not limited to standard metered dose inhalers or breath operated inhalers. Breath-actuated devices are also known, and have been the subject of many patent applications. Thus, for example, GB 1288971; GB 1297993; GB 1335378; GB 1383761; GB 1392192; GB 1413285; WO85/01880; GB 2204799; U.S. Pat. No. 4,803,978 and EP 018628OA describe inhalation-actuated dispensing devices for use with a pressurized aerosol-dispensing container.

In a preferred embodiment of the invention, administration is effected by a means of a pump or squeeze-actuated nebulizer. In more preferred embodiments of the invention administration is effected by means of a metered dose inhaler or an aerosol dispenser.

Formulations of the present invention may conveniently be present in unit dosage form and may be prepared by conventional pharmaceutical techniques as discussed above. Such techniques include the step of bringing into association the Δ⁸ THC moiety and the pharmaceutical carrier(s) or excipient(s) In general the formulations are prepared by uniformly and intimately bringing into association the active ingredient with liquid carriers or finely divided solid carriers or both, and then, if necessary, shaping the product.

Formulations-suitable for administration by inhalation includes formulations of Δ⁸ THC, in a form that can be dispensed by such inhalation devices known to those in the art. Such formulations may include carriers such as powders and aerosols. The inhalant compositions used in the present invention may comprise liquid or powdered compositions containing the active ingredient that are suitable for nebulization and intrabronchial use, or aerosol compositions administered via an aerosol unit dispensing metered doses.

Aerosol formulations for use in the subject method would typically include in addition to a therapeutically effective amount of a Δ⁸ THC moiety and at least one propellant. The formulations of the inventions may be solutions or suspensions of the Δ⁸ THC moieties.

Those of skill will appreciate that the amount of the Δ⁸ THC moiety may be tailored based on the solubility of the active ingredients, stability, commercial necessities, and medical requirements. Preferred formulations comprise from about 0.01 to about 10% of Δ⁸ THC moiety. More preferred formulations include from about 0.05 to about 6%. Δ⁸ THC moieties according to both aspects of the invention have been prepared from natural CBD by cyclization and purified by chromatography (see e.g., Abrahamov et al, supra). Preferably the Δ⁸ THC moiety is synthesized to a acceptable pharmaceutical purity (greater than 99% pure).

Preferred propellants include hydrofluoroalkanes (HFAs; e.g., HFA 134a, HFA 227, or a blend thereof) or chlorofluorocarbons (CFCs).

In some embodiments, the formulation includes additional active components such as, forexample, another cannabinoid. In particularly preferred embodiments, the additional cannabinoid is cannabidiol (CBD). CBD is commercially available. optionally, the formulations may contain surfactants and co-solvents and may be filled into conventional aerosol containers that are closed by a suitable metering valve. In a particularly preferred embodiment, the formulation may include ethanol.

The following non-limiting examples of formulations are representative for the purposes of illustration only:

| Δ⁸ THC moiety | CBD | Ethanol | HFA 134a | HFA 227 |
|---|---|---|---|---|
| % (w/w) | % (w/w) | % (w/w) | % (w/w) | % (w/w) |
| 0.02 | 0 | 0 | 99.98 | 0 |
| 0.02 | 0 | 2.0 | 0 | 97.98 |
| 0.05 | 0.05 | 0 | 99.9 | 0 |
| 1.0 | 0.5 | 10.0 | 48.5 | 40.0 |

The formulations according to the invention may optionally include any of the well known pharmaceutically acceptable carriers including diluents and excipients (see Remington's Pharmaceutical Sciences, 18^{th} Ed., Gennaro, Mack Publishing Co., Easton, Pa. (1990) and Remington: The Science and Practice of Pharmacy, Lippincott, Williams & Wilkins (1995).

Suitable liquid compositions comprise the active ingredient in an aqueous, pharmaceutically acceptable inhalant solvent, e.g., isotonic saline or bacteriostatic water. The solutions are administered by means of a pump or squeeze-activated nebulized spray dispenser, or by any other conventional means for causing or enabling the requisite dosage amount of the liquid composition to be inhaled into the patient's lungs.

Suitable powder compositions include, by way of illustration; powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule which may be inserted by the patient into a device that punctures the capsule and blows the powder out in a steady stream suitable for inhalation.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels, lotions, and pastes comprising the ingredient to be administered in a pharmaceutical acceptable carrier. A preferred topical delivery system is a transdermal patch containing the ingredient to be administered.

Formulations for rectal administration may be prepared as a suppository with a suitable base comprising, such as, for example, cocoa butter.

Formulations for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations wherein the carrier is a liquid, for administration for example via a nasal spray, aerosol, or as nasal drops, include aqueous or oily solutions of the active ingredient.

Formulations suitable for vaginal administration may be presented as pessaries, suppositories, tampons, creams, gels, pastes, foams or spray formulations containing, in addition to the active ingredients, such carriers as are known in the art to be appropriate.

Formulations suitable for parental administration include aqueous and non-aqueous sterile, injection solutions which may contain antioxidants, stabilizers, buffers, bacteriostats, and

In a second aspect, the invention provides methods for treating a mammal to alleviate the symptoms associated with a number of disease states. Therefore, an object of the invention is (a) the reduction, elimination or prevention of pain associated with any medical condition; (b) the stimulation of appetite; (c) the reduction, elimination or prevention of nausea; (d): the reduction, elimination or prevention of vomiting (anti-emetic properties.); (e) the relaxation of muscle tissue (e.g., for the treatment of multiple sclerosis).

The term “mammal” is used to designate any warm-blooded animal. Accordingly, the invention is useful for medical as well as veterinary uses.

The formulations used are as described for the first aspect of the invention. Therapeutically effective amounts of the formulations are administered to mammals potentially benefiting from treatment with a Δ⁸ THC moiety for a therapeutically effective period of time. Dosages will depend on the condition being treated, the particular compound, and other clinical factors such as weight and condition of the mammal and the route of administration.

The term “therapeutically effective amount” and “therapeutically effective period of time” are used to denote treatments at dosages and for periods effective to achieve the therapeutic result sought. Furthermore, one of skill will appreciate that the therapeutically effective amount of Δ⁸ THC moiety may be lowered or increased by fine tuning and altering the amount of the other component. The invention therefore provides a method to tailor the administration/treatment to the particular exigencies specific to a given mammal. Therapeutically effective ranges may be easily determined for example empirically by starting at relatively low amounts and by step-wise increments with concurrent evaluation of inhibition.

### EXAMPLES

### Example I

To identify dose-limiting toxicity, healthy human volunteers are administered Δ⁸ Tetrahydrocannibinol aerosol formulations according to the invention at low dosages which are incrementally escalated while monitoring the subjects for dose-limiting side effects (such as psychotropic symptoms)

### Example II

To identify therapeutically effective amounts and times, terminal oncology patients are administered Δ⁸ Tetrahydrocannibinol aerosol formulations according to the invention at low dosages which are incrementally escalated until either the maximum acceptable level in Example I is reached, or the side effects in patients become too high, or sufficient efficacy is seen that increasing the dose further is unnecessary.

The following examples illustrate alternative aerosol formulations.

### Example 1

| Ingredient | Weight in g |
|---|---|
| Ethanol | 0.10 |
| P-134a | 2.02 |
| delta-8-THC | 0.01 |
| Lipoid S100 ™ | 0.05 |

Lipoid S100™ is a phospholipid.

### Example 2

| Ingredient | Weight in g |
|---|---|
| Ethanol | 0.09 |
| P-134a | 1.83 |
| delta-8-THC | 0.01 |
| Brij ™ | 0.02 |

Brij™ is a Lauryl Polyoxyethylene

### Example 3

| Ingredient | Weight in g |
|---|---|
| Ethanol | 0.20 |
| P-134a | 3.80 |
| delta-8-THC | 0.01 |
| Salbutamol Sulphate | 0.01 |

1. Use of Δ⁸ Tetrahydrocannabinol in the manufacture of an aerosol formulation for medicinal administration to a patient from an aerosol delivery device.

2. Use as claimed in claim 1, in which the patient is suffering from a condition selected from pain, nausea, vomiting, appetite loss, multiple sclerosis and asthma.

3. Use as claimed in claim 2, in which the patient is a cancer patient undergoing chemotherapy, and the condition is selected from pain, nausea, vomiting and appetite loss.

4. Use as claimed in claim 2, in which the condition is pain associated with phantom limb syndrome.

5. Use as claimed in claim 2, in which the condition is appetite loss associated with anorexia nervosa.

6. Use as claimed in any one of claims 1 to 5, in which the aerosol formulation is for administration to the lungs of the patient.

7. Use as claimed in any one of claims 1 to 5, in which the aerosol formulation is for administration to the buccal or nasal mucosa of the patient.

8. An aerosol delivery device containing an aerosol formulation comprising Δ⁸ Tetrahydrocannabinol.

9. A device as claimed in claim 8, which is a metered dose inhaler and in which the aerosol formulation further comprises a propellant.

10. A device as claimed in claim 9, in which the propellant is selected from 1,1,1,2-tetrafluoroethane and 1,1,1,2,3,3,3-heptafluoropropane.

11. A device as claimed in claim 9 or claim 10, in which the aerosol formulation further comprises ethanol as a solvent.

12. An aerosol formulation for use in an aerosol delivery device, which comprises Δ⁸ Tetrahydrocannabinol.

13. An aerosol formulation as claimed in claim 12, which further comprises a propellant.

14. An aerosol formulation as claimed in claim 13, in which the propellant is selected from 1,1,1,2-tetrafluoroethane and 1,1,1,2,3,3,3-heptafluoropropane.

15. An aerosol formulation as claimed in claim 13 or claim 14, which further comprises ethanol as a solvent.

16. A method of treating a mammal suffering from a condition indicating treatment with a Δ⁸ Tetrahydrocannabinol, which comprises administering an aerosolized aerosol formulation containing a therapeutically effective amount of Δ⁸ Tetrahydrocannabinol to the mammal.

17. A method as claimed in claim 16, in which the mammal is suffering from a condition selected from pain, nausea, vomiting, appetite loss, multiple sclerosis and asthma.

18. A method as claimed in claim 17, in which the mammal is a cancer patient undergoing chemotherapy, and the condition is selected from pain, nausea, vomiting and appetite loss.

19. A method as claimed in claim 17, in which the condition is pain associated with phantom limb syndrome.

20. A method as claimed in claim 17, in which the condition is appetite loss associated with anorexia nervosa.

21. A method as claimed in claim in 16, in which the aerosolized aerosol formulation is administered to the lungs of the mammal.

22. A method as claimed in claim in 16, in which the aerosolized aerosol formulation is administered to the buccal or nasal mucosa of the mammal.
